(19) United States
(12) Patent Application Publication
(10) Pub. No.: US 2003/0018382 A1
(43) Pub. Date: Jan. 23, 2003

(54) Process for improving vision

(76) Inventors: Stephen C. Pflugfelder, Houston, TX (US); Ronald L. Gross, Houston, TX (US)

Correspondence Address: BAKER BOTTS LLP, C/O INTELLECTUAL PROPERTY DEPARTMENT, THE WARNER, SUITE 1300, 1299 PENNSYLVANIA AVE, NW, WASHINGTON, DC, 20004-2400, US

(21) Appl. No.: 10/174,983

(22) Filed: Jun. 20, 2002

Related U.S. Application Data
(60) Provisional application No. 60/305,618, filed on Jul. 17, 2001.

(51) Int. Cl. A61F 2/14

(52) U.S. Cl. 623/5.11; 623/5.16

(57) ABSTRACT

A process for improving the vision of a patient's eye, in which the patient's eye includes a cornea includes the step of instilling a bio-compatible material to the cornea of the eye, in which the bio-compatible material includes bio-compatible molecules and the cornea has a first composite refractive index and a first optical power. In addition, the bio-compatible molecules alter the first composite refractive index to a second composite refractive index and the first optical power to a second optical power. In another embodiment, a process for treating presbyopia of a patient's eye, in which the patient's eye includes a cornea, is described. The process includes the step of instilling a bio-compatible molecule to the cornea of the eye, in which the bio-compatible material includes bio-compatible molecules and the cornea has a first composite refractive index and a first optical power. In addition, the bio-compatible molecules alter the first composite refractive index to a second composite refractive index and the first optical power to a second optical power.

The present application claims priority from U.S. Provisional Patent Application No. 60/305,618, entitled “Process for Improving Vision,” the disclosure of which is incorporated herein by reference in its entirety.

### BACKGROUND OF THE INVENTION

1. Field of the Invention

The invention relates generally to a process for improving vision. Specifically, the invention is directed to a process for improving vision of a patient's eye by instilling a bio-compatible molecule to the cornea of the patient's eye.

2. Description of Related Art

Ametropia is a generic term which refers to any condition of imperfect refraction in the eyes, such as nearsightedness, farsightedness, presbyopia, or any astigmatism. It is known in the art that refraction of the eyes may be manipulated by altering the curvature of the cornea of the eye. For example, contact lenses may be effective in correcting the effects of an ametropia by manipulating the effective curvature of the cornea of the eye. However, a disadvantage of contact lenses, whether hard or soft contact lenses, is that wearers may experience a degree of discomfort when the lenses are being worn. In addition, contact lenses may be difficult to insert or remove from the eye, and also may be easily lost or misplaced. Glasses also may be effective in correcting the effects of an ametropia by manipulating the effective curvature of the eye. However, glasses may be easily lost, misplaced or broken, and also may be cumbersome to wear. In addition, if a wearer of the contact lenses or glasses forgets to wear the contact lenses or forgets to bring the glasses with them when they travel, they may suffer from the effects of an ametropia during the time that they are not wearing or able to wear the contact lenses or glasses.

Various surgical procedures also may be available to correct an ametropia by manipulating the curvature of the corneal lens of the eye. However, even the simplest surgical procedure presents some risk of permanent damage due to error or infection to a patient undergoing the surgical procedure. Moreover, such surgical procedures may be expensive, and the patient is likely to undergo post surgical discomfort and also may continue to experience discomfort, such as irritation of the eye, well after the completion of the surgical procedure.

Various corneal implant lenses also may be available to correct an ametropia by manipulating the effective curvature of the lens of the eye. However, such corneal implants involve surgical implantation, which presents some risks of permanent damage to the eye.

### SUMMARY OF THE INVENTION

Therefore, a need has arisen for a process that overcomes these and other shortcomings of the related art. A technical advantage of the present invention is that patients may improve their vision without having to undergo surgical procedures and, in particular, without having to receive corneal implants. Another technical advantage is that patients may improve their vision without using contact lenses or glasses. Yet another technical advantage of the present invention is that patients may temporarily improve their vision when they forget or lose their contact lenses or glasses, or if they do not wish to wear their contact lenses or glasses for a predetermined length of time.

In an embodiment of the present invention, a process for improving the vision of a patient's eye, in which the patient's eye includes a cornea, is described. The process includes the step of instilling a bio-compatible material to the cornea of the eye, in which the cornea has a first composite refractive index and a first optical power, and the bio-compatible material comprises a plurality of bio-compatible molecules. In addition, the bio-compatible molecules alter the first composite refractive index to a second composite refractive index and the first optical power to a second optical power.

In another embodiment of the present invention, a process for improving the vision of a patient's eye, in which the patient's eye includes a cornea, is described. The process includes the step of instilling a bio-compatible material to the cornea of the eye, in which the cornea has a first composite refractive index and a first optical power, and the bio-compatible material comprises a plurality of bio-compatible molecules. In addition, the bio-compatible molecules alter the first composite refractive index to a second composite refractive index and the first optical power to a second optical power for a predetermined length of time.

In yet another embodiment of the present invention, a process for treating presbyopia, nearsightedness, or farsightedness of a patient's eye, in which the patient's eye includes a cornea, is described. The process includes the step of instilling a bio-compatible material to the cornea of the eye, in which the cornea has a first composite refractive index and a first optical power, and the bio-compatible material comprises a plurality of bio-compatible molecules. In addition, the bio-compatible molecules alter the first composite refractive index to a second composite refractive index and the first optical power to a second optical power.

Other objects, features, and advantages will be apparent to persons of ordinary skill in the art in view of the following detailed description of the invention and the accompanying drawings.

### BRIEF DESCRIPTION OF THE DRAWINGS

For a more complete understanding of the present invention, the needs satisfied thereby, and the features and advantages thereof, reference now is made to the following descriptions taken in connection with accompanying drawings.

FIG. 1 is schematic of a cornea of a patient's eye according to an embodiment of the present invention.

FIG. 2 is a schematic of a process according to an embodiment of the present invention.

FIG. 3 is a chart depicting the results of an experiment conducted according to the process of FIG. 2.

### DETAILED DESCRIPTION OF PREFERRED EMBODIMENTS

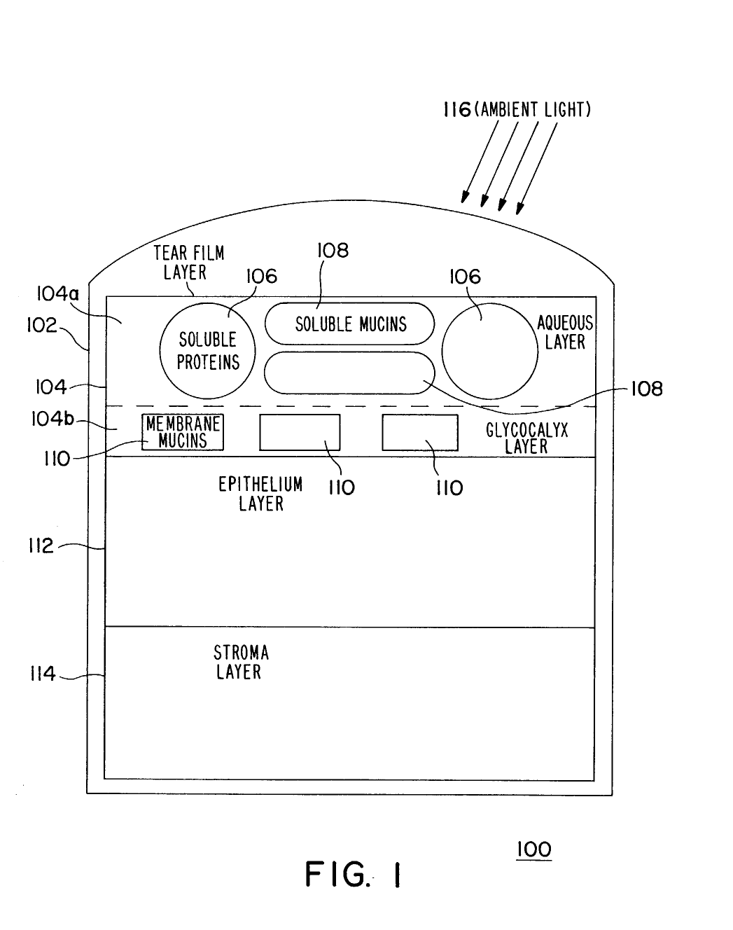

Preferred embodiments of the present invention and their advantages may be understood by referring to FIGS. 1-3, like numerals being used for like corresponding parts in the various drawings.

Referring to FIG. 1, a schematic for a patient's eye 100 is described. A patient 101 may have eye 100, which may comprise a cornea 102 and an inferior conjunctive sac or lower eye-lid 103. Cornea 102 may comprise a tear film layer 104, an epithelium layer 112, and a stroma layer 114. While not wishing to be bound by a theory, it is believed that tear film layer 104 may comprise an aqueous or tear mucin layer 104a and a glycocalyx layer or coating 104b. Moreover, aqueous layer 104a may comprise a plurality of soluble proteins 106 and a plurality of soluble mucins 108, and glycocalyx layer 104b may comprise a plurality of membrane mucins 110. For example, See, e.g., Stephen C. Pflugfelder et al., Detection of Sialomucin Complex (MUC4) in Human Ocular Surface Epithelium and Tear Fluid, Investigative Ophthalmology & Visual Science, May 2000, Volume 41, No. 6, at 1316, the disclosure of which is incorporated herein by reference in its entirety. In this embodiment, ambient light 116 first may enter cornea 102 through tear film layer 104.

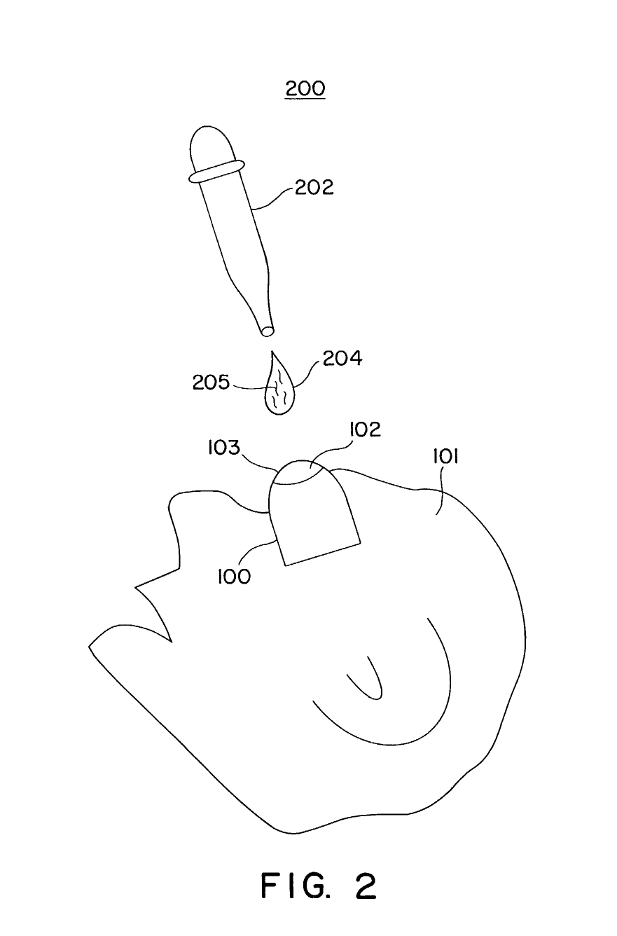

Referring to FIG. 2, a schematic of a process 200 for improving the vision of eye 100 according to an embodiment of the present invention is described. The process comprises the step of instilling a bio-compatible material 204 to cornea 102 of eye 100. It will be understood by those of ordinary skill in the art that the term “instill” may include any known means for contacting bio-compatible material 204 with cornea 102 of eye 100, such as via an eye dropper 202, an aerosol sprayer (not shown), or the like. Moreover, the term “bio-compatible” may include, any material which may be suitable for instillation in eye 100. Bio-compatible material 204 may comprise a plurality of bio-compatible molecules 205 and at least a portion of bio-compatible material 204 may be instilled on inferior conjunctive sac 103 by eye dropper 202, such that at least a portion of bio-compatible molecules 205 may permeate tear film 104. In an embodiment, bio-compatible material 204 may be a hydrophilic polyanionic diaccharide, such as a sodium hyaluronate gel. Nevertheless, it will be understood by those of ordinary skill in the art that bio-compatible material 204 may be any bio-compatible material which may alter the composite refractive index of cornea 102, e.g., a bio-compatible material comprising bio-compatible molecules which may form a chemical attraction with molecules in tear film 104, such as glycocalyx or epithelial cells. For example, the sodium hyaluronate gel may be about 1% sodium hyaluronate gel and about 50 μl of the 1% sodium hyaluronate gel may be instilled on inferior conjunctive sac 103. In an embodiment of the invention, the 1% sodium hyaluronate gel may be Healon® gel, which manufactured by Pharmacia of Peapack, N.J.

In addition, cornea 102 may have a first composite refractive index and a first optical power. The first composite refractive index of cornea 102 may be a summation of the refractive indices of the various layers of cornea 102. For example, the refractive index of tear film layer 104 may be between about 1.3357 and about 1.3374, and the refractive index of epithelium layer 112 and stroma layer 114 may be about 1.3760. In this embodiment, the first composite refractive index of cornea 102 may be about 1.3760. Moreover, the first optical power of cornea 102 may be related to the first composite index of refraction of cornea 102. For example, the first optical power of cornea 102 may be estimated using the formula:

Optical Power=(A−B)/C

in which A is the first composite refractive index of cornea 102, B is the refractive index of air, i.e., one (1), and C is the radius of curvature of the anterior corneal surface (not shown). In this embodiment, when the radius of curvature of the anterior corneal surface is about 7.8 mm, the first optical power may be estimated at about 48.2 diopters, i.e., (1.3760−1)/0.0078=48.2 diopters. However, although the estimated first optical power may be estimated at about 48.2 diopters, the actual or effective first optical power only may be about 43 diopters. The actual first optical power may be less than the estimated first optical power because there may be about a negative six (−6) diopter lens effect between the posterior cornea (not shown) and the aqueous humor (not shown).

When a measured amount of bio-compatible material 204 comprising the plurality of bio-compatible molecules 205 is instilled in eye 100, bio-compatible molecules 205 may alter a first refractive index of tear film 104 to a second refractive index. For example, while not wishing to be bound by a theory, it is believed that tear film 104 may comprise tear mucin layer 104a and glycocalyx layer 104b. Moreover, tear mucin layer 104a may comprise a plurality of molecules, such as proteins 106 and soluble mucins 108, which may bind bio-compatible molecules 205 to tear film 104. Similarly, glycocalyx layer 104b may comprise membrane mucins 110, which also may bind bio-compatible molecules 205 to tear film 104. Moreover, bio-compatible molecules 205 may have a refractive index which is greater than or less than the refractive index of tear film 104. When bio-compatible molecules 205 bind to tear film 104, because the refractive index of bio-compatible molecules 205 are different than the refractive index of tear film 104, the refractive index of tear film 104 may be altered.

Moreover, altering the refractive index of tear film 104 also may alter the first composite refractive index of cornea 102 to a second composite refractive index. Because the optical power of cornea 102 may be related to the composite refractive index of cornea 102, e.g., the estimated optical power may equal a difference between a composite refractive index of cornea 102 and the refractive index of air divided by the radius of curvature of the anterior corneal surface (not shown), altering the first composite refractive index of cornea 102 to a second composite refractive index also may alter the first optical power of cornea 102 to a second optical power.

For example, the refractive index of bio-compatible molecules 205 may be greater than the refractive index of tear film 104. When bio-compatible molecules 205 bind to tear film 104, because the refractive index of bio-compatible molecules 205 is greater than the refractive index of tear film 104, the refractive index of tear film 104 may be increased. Moreover, increasing the refractive index of tear film 104 also may increase the first composite refractive index of cornea 102 to a second composite refractive index. Because the optical power of cornea 102 is related to the composite refractive index of cornea 102, increasing the first composite refractive index of cornea 102 to a second composite refractive index also may increase the first optical power of cornea 102 to a second optical power. Alternatively, the refractive index of bio-compatible molecules 205 may be less than the refractive index of tear film 104, which may decrease the first composite refractive index of cornea 102 to a second composite refractive index and the first optical power of cornea 102 to a second optical power.

In addition, altering the first composite refractive index of cornea 102 to a second composite refractive index, may increase an ability of eye 100 to focus, which may improve the vision of eye 100. While not wishing to be bound by a theory, it is believed when patient 101 suffers from an ametropia of eye 100, ambient light 116 may not focus on a retina (not shown) of eye 100. For example, if patient 101 suffers from presbyopia or farsightedness, ambient light 116 may focus behind the retina of eye 100. If patient 101 suffers from nearsightedness, ambient light 116 may focus in front of the retina of eye 100. Specifically, ambient light 116 may include portions of light having varying wavelengths, such that the individual portions of ambient light 116 may enter eye 100 at different locations. Because cornea 102 may have an index of refraction which is different than the index of refraction of air, when ambient light 116 enters cornea 102, ambient light 116 may bend. Moreover, the individual portions of ambient light 116 may bend, such that the individual portions of ambient light 116 may converge and intersect at a single focal point. When the natural focal point of patient 101 is a location other than the retina of eye 100, the vision of patient 101 may be imperfect or at least slightly blurred. Altering the first composite index of cornea 102 to the second composite index may alter the focal point of eye 100, such that the focal point may be positioned on the retina of eye 100, which may improve the vision of patient 101.

In one embodiment, if patient 101 suffers from presbyopia, farsightedness, or another ametropia in which the focal point of eye 100 is located behind the retina of eye 100, increasing the first composite refractive index of cornea 102 to a second composite refractive index may increase an ability of eye 100 to focus, which may improve the vision of eye 100. Alternatively, if patient 101 suffers from nearsightedness or another ametropia in which the focal point of eye 100 is located in front of the retina of eye 100, decreasing the first composite refractive index of cornea 102 to a second composite refractive index may increase an ability of eye 100 to focus, which may improve the vision of eye 100. Moreover, it will be understood by one of ordinary skill in the art that whether increasing the first composite index of cornea 102 to the second composite index or decreasing the first composite index of cornea 102 to the second composite index will increase the ability of eye 100 focus, may depend on the particular ametropia from which patient 101 suffers.

The measured amount of bio-compatible material 204 containing bio-compatible molecules 205 instilled in eye 100 may vary depending on the particular ametropia from which patient 101 suffers and the severity of the ametropia. For example, a patient suffering from an ametropia which is more sever than an ametropia suffered by another patient, may require a greater amount of bio-compatible material 204 be instilled in eye 100 in order to raise the first composite refractive index of cornea 102 to a second composite index sufficient to position the focal point on the retina of eye 100, such that the ability of the patient to focus may increase to a desired level. For example, in one embodiment, patient 101 may suffer from presbyopia, such that the focal point is positioned behind the retina of eye 100, and cornea 102 may have a first composite index of refraction of about 1.3760. In this embodiment, raising the first composite refractive index of cornea 102 to a second composite refractive index of between about 1.3850 and about 1.4000 may be sufficient to position the focal point on the retina of eye 100. However, it will understood by one of ordinary skill in the art that the measured about of bio-compatible material 204 which may be used to position the focal point on the retina of eye 100 may vary from individual to individual and may be determined by individual testing and experimentation.

In another embodiment of the present invention, bio-compatible molecules 205 may alter the first composite refractive index of cornea 102 to the second composite refractive index for a predetermined length of time. Because the optical power of cornea 102 may be related to the composite refractive index of cornea 102, altering the first composite refractive index of cornea 102 to a second composite refractive index for a predetermined length of time also may alter the first optical power of cornea 102 to a second optical power for the predetermined length of time.

While not wishing to be bound by a theory, it is believed that the predetermined length of time may depend on such factors as a temperature and a humidity of an area where patient 101 located when is instilling bio-compatible material 204 into eye 100. For example, if the temperature or the humidity is greater at a first location than at a second location, bio-compatible material 204 may evaporate or may be excreted from the eye in the form of sweat more rapidly at the first location than at the second location. The predetermined length of time also may depend on such factors as the amount bio-compatible material 204 instilled in eye 100 and the actions of patient 101, e.g., physical activity, a rubbing or a blinking of eye 100, or a tearing of eye 100 may decrease the predetermined length of time. The predetermined length of time may be between about five (5) minutes and one (1) hour. In an embodiment, bio-compatible molecules 205 may increase the first composite refractive index of cornea 102 to the second composite refractive index and also may increase the first optical power of cornea 102 to the second optical power for the predetermined length of time. When patient 101 suffers from presbyopia or farsightedness, increasing the first composite refractive index of cornea 102 to a second composite refractive index may increase an ability of eye 100 to focus for the predetermined length of time, which may improve the vision of eye 100 for the predetermined length of time.

Alternatively, bio-compatible molecules 205 may decrease the first composite refractive index of cornea 102 to the second composite refractive index and also may decrease the first optical power of cornea 102 to the second optical power for the predetermined length of time. When patient 101 suffers from nearsightedness, decreasing the first composite refractive index of cornea 102 to a second composite refractive index may increase an ability of eye 100 to focus for the predetermined length of time, which may improve the vision of eye 100 for the predetermined length of time. However, it will be understood by one of ordinary skill in the art that whether increasing the first composite index of cornea 102 to the second composite index or decreasing the first composite index of cornea 102 to the second composite index will increase the ability of eye 100 focus, may depend on the particular ametropia which patient 101 suffers from.

### EXAMPLE

Embodiments of the present invention will be further clarified by consideration of the following example of an experiment conducted in accord with the above-described embodiments, which is intended to be purely exemplary of the use of the invention.

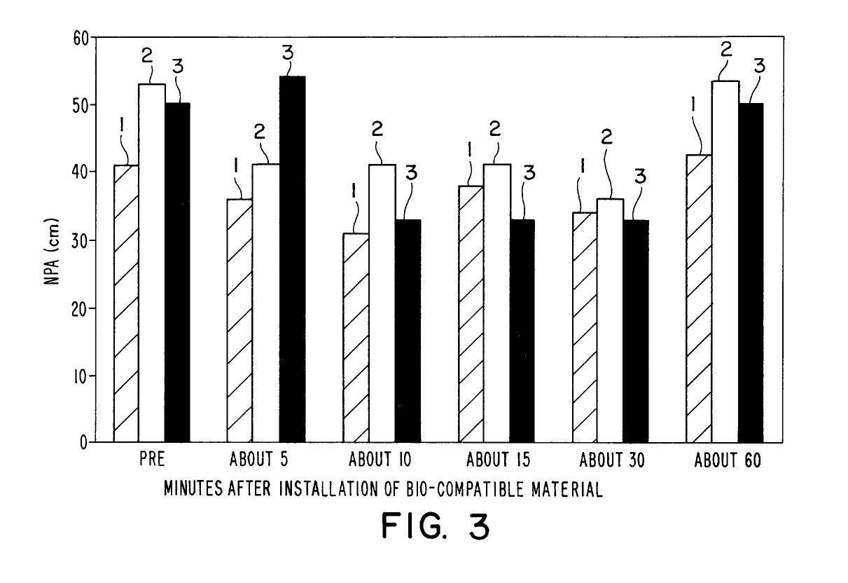

Referring to FIG. 3, an experiment for treating presbyopia according to the above described embodiments was conducted. The conducted experiment involved three subject individuals, a first patient 1, a second patient 2, and a third patient 3, respectively. Initially, the near point of accommodation (NPA), i.e., the point at which a target appears blurred to the individual, was calculated for each of the patients by advancing a fixation target towards the patient and asking them to report when the target appeared blurred. The NPA then was recorded in centimeters and the NPA was recalculated using the same process in order to confirm the accuracy of the calculation. Bio-compatible material 204, which was Healon® gel, then was instilled in conjunctival sac 103 using eye dropper 202 for each of the patients, and the patients were instructed to blink frequently for one (1) minute to facilitate permeation of bio-compatible molecule 204 to tear film 104. NPA measurements then were calculated at about five (5) minutes, about ten minutes, about thirty (30) minutes, and about sixty (60) minutes after the instillation of bio-compatible material 204. As the NPA of a particular patient decreases, the optical power of eye 100 will increase.

As shown in FIG. 3, after about five (5) minutes, patient 1 and patient 2 each experienced a decrease in their NPA, while patient 3 experienced a slight increase in NPA. After about ten (10) minutes, patient 1 experienced a further decrease in NPA, patient 2 experienced substantially no change in NPA between about five (5) and about ten (10) minutes, and patient 3 experienced a decrease in NPA. After about fifteen (15) minutes, patient 1 experienced a slight increase in NPA between about ten (10) and about fifteen (15) minutes and patient 2 and patient 3 experienced substantially no change in NPA between about ten (10) and about fifteen (15) minutes. After about thirty (30) minutes, patient 1 and patient 2 experienced a further decrease in NPA between about fifteen (15) minutes and about thirty (30) minutes, while patient 3 experienced substantially no change between about fifteen (15) minutes and about thirty (30) minutes. Finally, after about sixty (60) minutes, the NPA for each of the patients returned to about the same distance that was calculated for each of the respective patients prior to the instillation of bio-compatible material 204. In this experiment, patient 1 experienced maximum decrease in NPA relative to the NPA calculated prior to instillation of bio-compatible molecule 204 at about ten (10) minutes and patient 2 and patient 3 each experienced their maximum decrease in NPA at about thirty (30) minutes.

While the invention has been described in connecting with preferred embodiments, it will be understood by those of ordinary skill in the art that other variations and modifications of the preferred embodiments described above may be made without departing from the scope of the invention. Other embodiments will be apparent to those of ordinary skill in the art from a consideration of the specification or practice of the invention disclosed herein. It is intended that the specification and the described examples are considered as exemplary only, with the true scope and spirit of the invention indicated by the following claims

What is claimed is:

1. A process for improving the vision of a patient's eye, wherein said patient's eye comprises a cornea and said process comprises the step of:
instilling a bio-compatible material comprising a plurality of bio-compatible molecules to said cornea of said eye, wherein said cornea has a first composite refractive index and a first optical power, and said bio-compatible molecules alter said first composite refractive index to a second composite refractive index and said first optical power to a second optical power.

2. The process of claim 1, wherein said bio-compatible molecules increase said first composite refractive index to said second composite refractive index and said first optical power to said second optical power.

3. The process of claim 2, wherein said bio-compatible molecules increase said first composite refractive index to said second composite refractive index and said first optical power to said second optical power for a predetermined length of time.

4. The process of claim 3, wherein said predetermined length of time is between about five (5) minutes and about one (1) hour.

5. The process of claim 4, wherein said first optical power is about 48.2 diopters.

6. The process of claim 2, wherein said bio-compatible material is a hydrophilic polyanionic disaccharide.

7. The process of claim 6, wherein said hydrophilic polyanionic disaccharide is a sodium hyaluronate gel.

8. The process of claim 7, wherein said sodium hyaluronate gel is about a 1% sodium hyaluronate gel.

9. The process of claim 8, wherein an amount of said 1% sodium hyaluronate gel instilled to said cornea is about 50 μl.

10. The process of claim 2, wherein said bio-compatible molecules increase an ability of said cornea to focus.

11. The process of claim 10, wherein said ability of said cornea to focus increases for between about five (5) minutes and about one (1) hour.

12. The process of claim 10, wherein said first composite refractive index is about 1.3760.

13. The process of claim 10, wherein said cornea comprises a tear film and at least a portion of said bio-compatible molecules permeate said tear film.

14. The process of claim 13, wherein said patient's eye further comprises an inferior conjunctival sac and at least a portion of said bio-compatible material is instilled on said inferior conjunctival sac.

15. The process of claim 13, wherein said tear film comprises a tear mucin layer, wherein said tear mucin layer binds said bio-compatible molecules to said cornea.

16. A process for improving a vision of a patient's eye, wherein said patient's eye comprises a cornea and said process comprises the step of:
instilling a bio-compatible material comprising a plurality of bio-compatible molecules to said cornea of said eye, wherein said cornea has a first composite refractive index and a first optical power, and said bio-compatible molecules alter said first composite refractive index to a second composite refractive index and said first optical power to a second optical power for a predetermined length of time.

17. The process of claim 16, wherein said bio-compatible molecules increase said first composite refractive index to said second composite refractive index and said first optical power to said second optical power for said predetermined length of time.

18. The process of claim 17, wherein said predetermined length of time is between about five (5) minutes and about one (1) hour.

19. The process of claim 18, wherein said first optical power is about 48.2 diopters.

20. The process of claim 17, wherein said bio-compatible material is a hydrophilic polyanionic disaccharide.

21. The process of claim 20, wherein said hydrophilic polyanionic disaccharide is a sodium hyaluronate gel.

22. The process of claim 21, wherein said sodium hyaluronate gel is about a 1% sodium hyaluronate gel.

23. The process of claim 22, wherein an amount of said 1% sodium hyaluronate gel instilled to said cornea is about 50 μl.

24. The process of claim 17, wherein said bio-compatible molecules increase an ability of said cornea to focus.

25. The process of claim 24, wherein said ability of said cornea to focus increases for between about five (5) minutes and about one (1) hour.

26. The process of claim 24, wherein said first composite refractive index is about 1.3760.

27. The process of claim 24, wherein said cornea comprises a tear film and at least a portion of said bio-compatible molecules permeate said tear film.

28. The process of claim 27, wherein said patient's eye further comprises an inferior conjunctival sac and at least a portion of said bio-compatible material is instilled on said inferior conjunctival sac.

29. The process of claim 27, wherein said tear film comprises a tear mucin layer, wherein said tear mucin layer binds said bio-compatible molecules to said cornea.

30. A process for treating presbyopia in a patient's eye, wherein said patient's eye comprises a cornea and said process comprises the step of:
instilling a bio-compatible material comprising a plurality of bio-compatible molecules to said cornea of said eye, wherein said cornea has a first composite refractive index and a first optical power, and said bio-compatible molecules alter said first composite refractive index to a second composite refractive index and said first optical power to a second optical power.

31. The process of claim 30, wherein said bio-compatible molecules increases said first composite refractive index to said second composite refractive index and said first optical power to said second optical power.

32. The process of claim 31, wherein said bio-compatible molecules increase said first composite refractive index to said second composite refractive index and said first optical power to said second optical power for a predetermined length of time.

33. The process of claim 32, wherein said predetermined length of time is between about five (5) minutes and about one (1) hour.

34. The process of claim 33, wherein said first optical power is about 48.2 diopters.

35. The process of claim 31, wherein said bio-compatible material is a hydrophilic polyanionic disaccharide.

36. The process of claim 35, wherein said hydrophilic polyanionic disaccharide is a sodium hyaluronate gel.

37. The process of claim 36, wherein said sodium hyaluronate gel is about a 1% sodium hyaluronate gel.

38. The process of claim 37, wherein an amount of said 1% sodium hyaluronate gel instilled to said cornea is about 50 μl.

39. The process of claim 31, wherein said bio-compatible molecules increase an ability of said cornea to focus.

40. The process of claim 39, wherein said ability of said cornea to focus increases for between about five (5) minutes and about one (1) hour.

41. The process of claim 39, wherein said first composite refractive index is about 1.3760.

42. The process of claim 39, wherein said cornea comprises a tear film and at least a portion of said bio-compatible molecules permeate said tear film.

43. The process of claim 42, wherein said patient's eye further comprises an inferior conjunctival sac and at least a portion of said bio-compatible material is instilled on said inferior conjunctival sac.

44. The process of claim 42, wherein said tear film comprises a tear mucin layer, wherein said tear mucin layer binds said bio-compatible molecules to said cornea.

45. A process for treating nearsightedness in a patient's eye, wherein said patient's eye comprises a cornea and said process comprises the step of:
instilling a bio-compatible material comprising a plurality of bio-compatible molecules to said cornea of said eye, wherein said cornea has a first composite refractive index and a first optical power, and said bio-compatible molecules alter said first composite refractive index to a second composite refractive index and said first optical power to a second optical power.

46. The process of claim 45, wherein said bio-compatible molecules decrease said first composite refractive index to said second composite refractive index and said first optical power to said second optical power.

47. The process of claim 46, wherein said bio-compatible molecules decrease said first composite refractive index to said second composite refractive index and said first optical power to said second optical power for a predetermined length of time.

48. The process of claim 47, wherein said predetermined length of time is between about five (5) minutes and about one (1) hour.

49. The process of claim 48, wherein said first optical power is about 48.2 diopters.

50. The process of claim 46, wherein said bio-compatible material is a hydrophilic polyanionic disaccharide.

51. The process of claim 50, wherein said hydrophilic polyanionic disaccharide is a sodium hyaluronate gel.

52. The process of claim 51, wherein said sodium hyaluronate gel is about a 1% sodium hyaluronate gel.

53. The process of claim 52, wherein an amount of said 1% sodium hyaluronate gel instilled to said cornea is about 50μl.

54. The process of claim 46, wherein said bio-compatible molecules increase an ability of said cornea to focus.

55. The process of claim 54, wherein said ability of said cornea to focus increases for between about five (5) minutes and about one (1) hour.

56. The process of claim 54, wherein said first composite refractive index is about 1.3760.

57. The process of claim 54, wherein said cornea comprises a tear film and at least a portion of said bio-compatible molecules permeate said tear film.

58. The process of claim 57, wherein said patient's eye further comprises an inferior conjunctival sac and at least a portion of said bio-compatible material is instilled on said inferior conjunctival sac.

59. The process of claim 57, wherein said tear film comprises a tear mucin layer, wherein said tear mucin layer binds said bio-compatible molecules to said cornea.

60. A process for treating farsightedness in a patient's eye, wherein said patient's eye comprises a cornea and said process comprises the step of:
instilling a bio-compatible material comprising a plurality of bio-compatible molecules to said cornea of said eye, wherein said cornea has a first composite refractive index and a first optical power, and said bio-compatible molecules alter said first composite refractive index to a second composite refractive index and said first optical power to a second optical power.

61. The process of claim 60, wherein said bio-compatible molecules increase said first composite refractive index to said second composite refractive index and said first optical power to said second optical power.

62. The process of claim 61, wherein said bio-compatible molecules increase said first composite refractive index to said second composite refractive index and said first optical power to said second optical power for a predetermined length of time.

63. The process of claim 62, wherein said predetermined length of time is between about five (5) minutes and about one (1) hour.

64. The process of claim 63, wherein said first optical power is about 48.2 diopters.

65. The process of claim 61, wherein said bio-compatible material is a hydrophilic polyanionic disaccharide.

66. The process of claim 65, wherein said hydrophilic polyanionic disaccharide is a sodium hyaluronate gel.

67. The process of claim 66, wherein said sodium hyaluronate gel is about a 1% sodium hyaluronate gel.

68. The process of claim 67, wherein an amount of said 1% sodium hyaluronate gel instilled to said cornea is about 50 μl.

69. The process of claim 61, wherein said bio-compatible molecules increase an ability of said cornea to focus.

70. The process of claim 69, wherein said ability of said cornea to focus increases for between about five (5) minutes and about one (1) hour.

71. The process of claim 69, wherein said first composite refractive index is about 1.3760.

72. The process of claim 69, wherein said cornea comprises a tear film and at least a portion of said bio-compatible molecules permeate said tear film.

73. The process of claim 72, wherein said patient's eye further comprises an inferior conjunctival sac and at least a portion of said bio-compatible material is instilled on said inferior conjunctival sac.

74. The process of claim 72, wherein said tear film comprises a tear mucin layer, wherein said tear mucin layer binds said bio-compatible molecules to said cornea.
